(19) United States
(12) Patent Application Publication
(10) Pub. No.: US 2005/0228317 A1
(43) Pub. Date: Oct. 13, 2005

(54) Warning device for prevention of deep vein thrombosis

(76) Inventor: Steven Charles Mathews, New York, NY (US)

Correspondence Address: ROBERT T. MALDONADO;Cooper & Dunham LLP, 1185 Avenue of the Americas, New York, NY, 10036, US

(21) Appl. No.: 10/816,145

(22) Filed: Apr. 1, 2004

(51) Int. Cl. A61B 5/103; A61B 5/117

(52) U.S. Cl. 600/595; 600/587

(57) ABSTRACT

A device for monitoring a length of time a person has been immobile is provided comprising a power supply unit, control unit, pressure sensor, and warning unit. The warning unit is triggered by the control unit to alert a person that he/she has been immobile for a prolonged period of time.

### BACKGROUND

1. Technical Field

The present disclosure relates generally to a device for monitoring the length of time that a person has been immobile in order to avoid deep vein thrombosis.

2. Description of the Related Art

Deep vein thrombosis (or “DVT”) is a blood clot (thrombus) that can develop in deep veins found in the body, usually in the thigh. This abnormal blood clot can interfere with blood circulation in the area or the clot can break off and travel into the blood stream. If the blood clot lodges itself in the brain, lungs, heart, or other organ, it can cause serious illness or even death, a condition known as pulmonary embolism.

According to the American Heart Association, an estimated two million Americans experience DVT each year. In fact, DVT is especially common among travellers, who are immobile for prolonged periods of time (for example, during long car trips or air travel). See American Heart Organization, Economy Class Syndrome and Deep Vein Thrombosis, available at http://www.americanheart.org/presenter.jhtml?identifier=3010041 (last visited on Mar. 3, 2004). Although DVT is also known as “economy class syndrome”, the name is a misnomer because DVT can affect all travellers, no matter what class of travel they use. When the human body is still for long periods of time, blood flow in the veins decreases, placing them at a greater risk of developing blood clots, and consequently, a dangerous pulmonary embolism.

Symptoms of DVT include swelling and tenderness of the affected area. However, quite often, DVT strikes without warning. In these cases, the first sign of DVT might be serious illness or death. Each year, as many as 200,000 people die from pulmonary embolism in the United States. See Schreiber, Donald, Deep Venous Thrombosis and Thrombophlebitis, eMedicine Journal (Jun. 26, 2001), available at http://www.emedicine.com/emerg/topic122.htm (last visited on Mar. 3, 2004). Given the gravity of this statistic and the seriousness of the disease, preventing DVT can avoid serious injuries and save lives.

According to a NASA Occupational Health Alert, it is recommended that a person stand up and walk around at least hourly during travel in order to prevent DVT. See NASA OH—Health Alerts, Flight-related Deep Vein Thrombosis (DVT)—Economy Class Syndrome, available at http://ohp.nasa.gov/alerts/dvt.html (last visited on Mar. 3, 2004).

The risk of DVT can be controlled by using electricity to stimulate the muscles in an area, or applying pressure to the area to assist in blood circulation. One such device is disclosed in U.S. Pat. No. 6,282,448 B1 issued to Katz et al. on Aug. 28, 2001. The Katz device electrically stimulates the nerves and muscles in an area to prevent DVT. Another device is disclosed in U.S. Pat. No. 6,290,662 B1 issued to Morris et. al on Sep. 18, 2001. The Morris device uses compression to apply pressure to an area to facilitate blood circulation to a particular part of a person's body. However, both of these prior art devices can be expensive to manufacture, uncomfortable to use, and inconvenient for travellers to carry and use.

The need therefore remains for a device that monitors the length of time that a person has been immobile to prevent DVT that is simple, inexpensive, convenient to transport, and comfortable to use. This invention meets that need and overcomes the disadvantages of the prior art described above.

### SUMMARY

The present invention overcomes the shortcomings of the prior art by providing a device for monitoring the length of time a person has been immobile to prevent DVT, according to one embodiment of the present invention, includes a power supply unit, a control unit, wherein the control unit is programmed to operate with associated hardware, a pressure sensor for detecting if a person has been immobile for a prolonged period of time, and a warning unit triggered by the control unit to generate a warning signal when a person has been immobile for a prolonged period of time.

A method for monitoring the length of time that a person has been immobile, according to one embodiment of the present invention, includes resetting a control unit, detecting whether a pressure sensor is activated, setting a first timer to a predetermined first time period, and activating a warning signal if the pressure sensor remains activated during the entire first time period.

A computer storage medium including computer executable code for monitoring the length of time that a person has been immobile, according to one embodiment of the present invention, includes code for resetting a control unit, code for monitoring a pressure sensor, code for setting a first timer to a predetermined first time period, and code for activating a warning signal if the pressure sensor remains activated during the entire first time period.

### BRIEF DESCRIPTION OF THE DRAWINGS

A more complete appreciation of the present invention and its advantages is readily obtained by reference to the following detailed description and accompanying drawings, wherein:

FIG. 1 shows a block diagram illustrating a stand alone device for monitoring the length of time a person has been immobile, according to one embodiment of the present invention; and

FIG. 2 shows a flow chart illustrating the execution of the device for monitoring the length of time a person has been immobile, according to one embodiment of the present invention.

### DETAILED DESCRIPTION

This application provides tools (in the form of methods, apparatuses, and systems) for preventing DVT. These tools may be embodied in one or more programs stored on a computer readable medium or program storage device and/or transmitted via a computer network or other transmission medium. Alternatively, the program can be stored and run on a microcontroller or microprocessor on a stand alone unit, a mobile phone, a personal digital assistant, or on a laptop or desktop computer.

The following exemplary embodiments are set forth to aid in an understanding of the subject matter of this disclosure, but are not intended, and should not be construed, to limit in any way the claims which follow thereafter. Therefore, while specific terminology is employed for the sake of clarity in describing some exemplary embodiments, the present disclosure is not intended to be limited to the specific terminology so selected, and it is to be understood that each specific element includes all technical equivalents which operate in a similar manner.

The device or method as provided herein can be used to monitor the length of time a person has been immobile and alert the person when the length of time that they have been immobile exceeds a predetermined amount, for example, one hour. A one hour time period is mentioned by NASA as a maximum suggested time period an individual should be immobile without getting up and moving about. Naturally, this time period can be other lengths of time, as this information is documented or discovered, and published. After being alerted that he or she has been immobile for greater than or equal to a first time period (e.g., greater than one hour), the person can immediately take appropriate action (for example, the person can get up, walk, stretch and move around), thereby minimizing the risk of DVT. The device can be utilized, for example, during air travel, on a long car ride, while watching television, or during a recovery period when a person is overcoming an illness.

The specific embodiments described herein are illustrative, and many variations can be introduced on these embodiments without departing from the spirit of the disclosure or from the scope of the appended claims. Elements and/or features of different illustrative embodiments may be combined with each other and/or substituted for each other within the scope of this disclosure and appended claims.

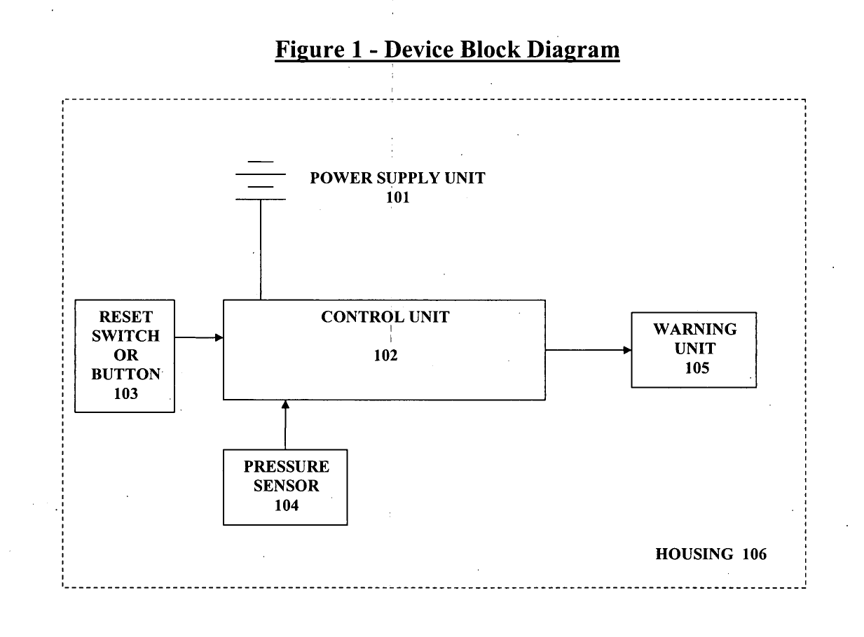

FIG. 1 is a block diagram illustrating a stand alone device for monitoring the length of time a person has been immobile, according to one embodiment of the present invention. The device comprises a power supply unit 101, control unit 102, reset switch or button 103, pressure sensor 104, warning unit 105, and housing 106. Power supply unit 101 supplies power to control unit 102. According to an embodiment of the present invention, the power supply unit 101 can be a battery. For example, if the device is used during a long car or plane ride, it might be desirable for the device to be battery-operated. On the other hand, if the device is being used in connection with prolonged bed rest, it might be desirable to be able to plug the device into a wall outlet.

Pressure sensor 104 can provide input to control unit 102. Pressure sensor 104 can be configured to detect if a person has been immobile for a prolonged period of time. For example, if a person is sitting, the device could be placed underneath the person's thigh, thereby activating the pressure sensor. When that person gets up, the pressure sensor would be de-activated. According to one embodiment of the present invention, control unit 102 can be a microcontroller, microprocessor or equivalent control circuit, each of which can be programmed to operate with the associated hardware (for example, pressure sensor 104). A reset switch or button 103 may also be provided in order to reset control unit 102. The control unit 102 can be the microprocessor or microcontroller residing in a mobile phone, personal digital assistant, laptop or desktop computer.

The output of control unit 102 can trigger the warning unit 105 to generate a warning signal to alert a person that they have been immobile for a prolonged period of time, for example, one hour. According to one embodiment of the present invention, the warning unit 105 can be a vibrating element, speaker or sound generating element, or light emitting diode or light bulb, and/or the vibrating element, the speaker or screen on a mobile phone, a personal digital assistant, laptop or desktop computer.

In the case of a stand alone device, a housing 106 can provide a protective outer shell that encloses power supply unit 101, control unit 102, reset switch or button 103, pressure sensor 104, and warning unit 105. A means can also be provided to connect the device to a person. For example, a strap, band or adhesive sensor can be used to attach the device to a specific portion of a person's body. According to another embodiment of the present invention, the pressure sensor 104 can be connected to the user with a strap, an adhesive patch, or velcro.

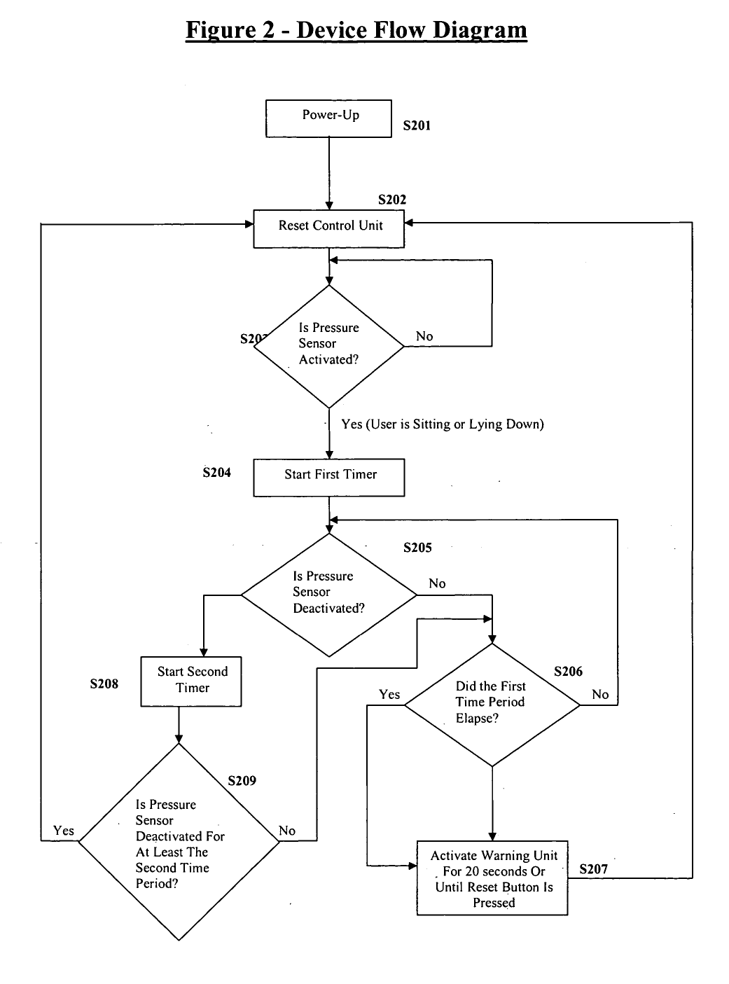

FIG. 2 is a flow chart illustrating the execution of the device for monitoring the length of time a person has been immobile, according to one embodiment of the present invention. The method set forth in this flow chart can be implemented on a stand alone device, or a mobile phone, personal digital assistant or laptop or desktop computer. The device is powered up (Step S201) and the control unit, for example, a microcontroller or microprocessor can be reset (Step S202). If the pressure sensor is activated (for example, a person is sitting down) (Step S203), a first timer is started, timing a first time period, for example, one hour (Step S204). If the pressure sensor is not activated, then the device waits for the sensor to be activated.

If the pressure sensor is activated (Step S205) and the first tinge period has not elapsed (Step S206), then the system will loop back and check to see if the pressure sensor is deactivated (Step S205) or the first time period has elapsed. If the pressure sensor is activated (Step S205) and the first time period has elapsed (Step S206), then the system will issue a warning signal (such as a vibrator, a buzzer, an LED, or a flashing screen on mobile phone or computing device such as personal digital assistant, laptop, or desktop computer) for a certain time, for example, twenty seconds, or until the reset button is pressed (Step S207).

Alternatively, if the pressure sensor is deactivated during the first time period, a second timer is triggered (Step S208), which measures the second time period, for example three minutes. If the pressure sensor remains deactivated during the entire length of the second time period (Step S209) (i.e., the user has gotten up for three minutes), then the control unit is reset and begins counting again (Step S202). Alternatively, if the pressure sensor is not deactivated for at least the second time period, then the device continues with the first timer and checks to see if the first time period has elapsed (Step S206).

Numerous additional modifications and variations of the present disclosure are possible in view of the above-teachings. It is therefore to be understood that within the scope of the appended claims, the present disclosure may be practiced other than as specifically described herein.

1. A device for monitoring a length of time a person has been immobile, comprising:
a power supply unit;
a control unit, wherein the control unit is programmed to operate with associated hardware;
a pressure sensor for detecting if a person has been immobile for a prolonged period of time; and
a warning unit triggered by the control unit to generate a warning signal when a person has been immobile for a prolonged period of time.

2. The device of claim 1, wherein the device is used to prevent deep vein thrombosis.

3. The device of claim 1, further comprising a housing that encloses the power supply unit, the control unit, the pressure sensor, and the warning.

4. The device of claim 1, wherein the pressure sensor is connected to the user with a strap, an adhesive patch, or velcro.

5. The device of claim 1, wherein the power supply unit is a battery.

6. The device of claim 1, wherein the control unit is a microcontroller, microprocessor or programmable electronic circuit.

7. The device of claim 1, wherein the warning unit is a vibrating element, a speaker or sound generating element, a light emitting diode (LED) or light bulb, or warning message on a screen or monitor.

8. A method for monitoring a length of time that a person has been immobile, comprising:
resetting a control unit;
detecting whether a pressure sensor is activated;
setting a first timer to a predetermined first time period; and
activating a warning signal if the pressure sensor remains activated during the entire first time period.

9. The method of claim 8, wherein the method is used to prevent deep vein thrombosis.

10. The method of claim 8, wherein the method is implemented on a stand alone device, a mobile phone, a personal digital assistant, or on a laptop or desktop computer.

11. The method of claim 8, further comprising setting a second timer to a second predetermined time period when the pressure sensor is deactivated.

12. The method of claim 11, wherein the first timer is reset if the pressure sensor is deactivated for a period of time greater than or equal to the second predetermined time period.

13. The method of claim 11, further comprising resetting the first timer if the pressure sensor is deactivated before the second time period elapses.

14. A computer storage medium, including computer executable code for monitoring a length of time that a person has been immobile, comprising:
code for resetting a control unit;
code for monitoring a pressure sensor;
code for setting a first timer to a predetermined first time period; and
code for activating a warning signal if the pressure sensor remains activated during the entire first time period.

15. The computer storage medium of claim 14, including computer executable code for monitoring a length of time that a person has been immobile to prevent deep vein thrombosis.

16. The computer storage medium of claim 14, wherein the computer is a stand alone device, a mobile phone, a personal digital assistant, or a laptop or desktop computer.

17. The computer storage medium of claim 14, further comprising code for setting a second timer to a second predetermined time period when the pressure sensor is deactivated.

18. The computer storage medium of claim 17, further comprising code for continuing with the first timer if the pressure sensor is deactivated for a period of time less than the second predetermined time period.
